(19) United States
(12) Patent Application Publication
(10) Pub. No.: US 2006/0093577 A1
(43) Pub. Date: May 4, 2006

(54) Combination anti-viral compositions and methods of use

(75) Inventor: Dominique Dugourd, Vancouver (CA)

Correspondence Address: DARBY & DARBY P.C., P. O. BOX 5257, NEW YORK, NY, 10150-5257, US

(73) Assignee: Migenix Inc., Vancouver (CA)

(21) Appl. No.: 11/244,811

(22) Filed: Oct. 6, 2005

Related U.S. Application Data
(60) Provisional application No. 60/616,787, filed on Oct. 6, 2004.

(51) Int. Cl. A61K 38/21; A61K 31/13

(52) U.S. Cl. 424/85.4; 424/85.7; 514/674

(57) ABSTRACT

The present disclosure relates generally to the use of castanospermine in combination with another therapeutic agent to treat or prevent infections caused by or associated with a virus of the Flaviviridae family, particularly infections caused by or associated with Hepatitis C virus (HCV), and to the use of such compounds to examine the biological mechanisms of HCV infection.

### CROSS-REFERENCE TO RELATED APPLICATIONS

This application claims the benefit of U.S. Provisional Patent Application No. 60/616,787, filed on Oct. 6, 2004, which provisional application is incorporated herein by reference in its entirety.

### TECHNICAL FIELD

The present disclosure relates generally to the treatment of infectious disease, and more specifically, the use of castanospermine in combination with an anti-viral compound to treat or prevent infections caused by or associated with Flaviviridae, particularly infections caused by or associated with hepatitis C virus (HCV).

### BACKGROUND

The flavivirus group includes the causative agents of numerous human diseases and a variety of animal diseases that cause significant losses to the livestock industry. The family Flaviviridae (members of which are referred to herein as flaviviruses) include the genera Flavivirus (e.g., yellow fever virus, dengue viruses, Japanese encephalitis virus, and tick-borne encephalitis virus); Pestivirus (e.g., bovine viral diarrhea virus (BVDV); classic swine fever virus, and border disease virus); Hepacivirus (e.g., hepatitis C virus); and currently unclassified members of the Flaviviridae (e.g., GB virus types A, B and C). Members of the Flaviviridae are described in detail by the International Committee on Taxonomy of Viruses (the currently accepted taxonomic definition is described in: Virus Taxonomy: The Classification and Nomenclature of Viruses. The Seventh Report of the International Committee on Taxonomy of Viruses, van Regenmortel et al., Academic Press, San Diego (2000)), the contents of which are hereby incorporated by reference).

A clinically significant member of the Flaviviridae family is hepatitis C virus (HCV). HCV was first identified in 1989 and is a major cause of acute hepatitis, responsible for most cases of post-transfusion non-A, non-B hepatitis. In addition, HCV is a major cause of chronic liver disease, including cirrhosis and liver cancer (Hooffnagle, Hepatology 26:15S, 1997). The World Health Organization estimates that 170 million people are chronically infected with HCV. Approximately 3-4 million people are newly infected each year, and 80-85% of these infected patients develop chronic infection with approximately 20-30% of these patients progressing to cirrhosis and end-stage liver disease, frequently complicated by hepatocellular carcinoma (HCC) (see, e.g., Kolykhalov et al., J. Virol. 74:2046, 2000).

HCV is an enveloped positive-strand RNA virus. The genome carries at the 5′ and 3′ ends non-translated regions (NTRs) that form stable secondary and tertiary structures. The 5′ NTR carries an internal ribosome entry site (IRES) permitting the direct binding of ribosomes in close proximity to the start codon of the open reading frame. Thus, translation of HCV RNA is mediated by the IRES, rather than the CAP-dependent mechanism typically used for translation of cellular mRNA.

The HCV genome consists of a single long open reading frame that encodes a polyprotein of approximately 3000 amino acid residues. This polyprotein is processed co- and post-translationally into at least 10 different products, including two N-linked glycosylated proteins E1 and E2. Within the polyprotein, cleavage products are ordered as follows: core (C); envelope protein 1 (E1); E2; p7 (which may be an ion channel-forming polypeptide); non-structural protein 2 (NS2); NS3; NS4A; NS4B; NS5A; and NS5B. The core protein is a highly basic RNA binding protein forming the major constituent of the nucleocapsid. The envelope proteins E1 and E2 are highly glycosylated type 1 membrane proteins anchored through the carboxy-terminal region. They are embedded into the lipid envelope of the virus particle and associate to form stable heterodimers. The non-structural proteins are involved in viral replication and possess protease (NS2/NS3), helicase (NS3), and RNA polymerase activities (NS5B). Binding of HCV to the host cell probably requires the interaction of E2 or the E1/E2 complex with a receptor that is present on the cell surface.

An understanding of the mechanism of HCV particle assembly is limited. The absence of complex glycans, the localization of expressed HCV glycoproteins in the endoplasmic reticulum (ER), and the absence of these proteins on the cell surface, together suggests that initial virion morphogenesis occurs by budding into intracellular vesicles from the ER. Additionally, mature E1-E2 heterodimers do not leave the ER, and ER retention signals have been identified in the C-terminal regions of both E1 and E2. Therefore, the virus would be exported via the constitutive secretory pathway. In agreement with this assumption, complex N-linked glycans were found on the surface of partially purified virus particles, suggesting that the virus transits through the Golgi.

Until recently, interferon-α (IFN-α) was the only therapy with proven benefit for the treatment of HCV infection. Approximately 50% of patients show an initial response to treatment with IFN-α, but the response is not sustainable in the majority of patients and the patients suffer considerable associated side effects. The current standard of care for treating HCV infection is administration of IFN-α with the nucleoside analogue ribavirin. However, identification of therapeutic candidates that have more potent antiviral activity and fewer undesirable side effects is needed.

Hence, a need exists for identifying and developing anti-Flaviviridae agents that have improved anti-viral activity and reduced toxicity, and in particular therapeutics for the treatment of HCV. The present invention meets such needs, and further provides other related advantages.

### SUMMARY

The present invention generally provides castanospermine compositions for use in treating or preventing, for example, Flaviviridae infections, such as those caused by hepatitis C virus (HCV). In particular, the present disclosure provides castanospermine in combination with other anti-Flaviviridae compounds, providing unexpectedly high or synergistic, inhibitory activity against HCV.

In one embodiment, the invention provides a method for treating a Flaviviridae infection comprising administering to a subject castanospermine, or a pharmaceutically acceptable salt thereof, and an agent that alters immune function. In another embodiment, a method is provided for treating a Flaviviridae infection comprising administering to a subject castanospermine, or a pharmaceutically acceptable salt thereof, and an agent that alters replication of a virus of the Flaviviridae family. In certain embodiments, the subject is a human. In other certain embodiments, the virus of the Flaviviridae family is a member of the genus Flavivirus, which may be a Hepacivirus, wherein the Hepacivirus is Hepatitis C virus (HCV), or the virus is a member of the genus Pestivirus. In another certain embodiment, the agent that alters immune function is an interferon, such as an interferon-α; and in a particular embodiment, the interferon-α is pegylated. The invention also provides a method wherein castanospermine and the agent that alters immune function are administered sequentially (wherein castanospermine is administered before the agent that alters immune function or wherein the agent that alters immune function is administered before castanospermine) or castanospermine and the agent are administered concurrently. In another embodiment, castanospermine and the agent that alters immune function are admixed as a single composition and administered concurrently. In another embodiment, the method comprises administering to a subject (a) a composition comprising castanospermine and a pharmaceutically acceptable carrier and (b) a composition comprising the agent that alters immune function and a pharmaceutically acceptable carrier.

In another embodiment, the agent that alters viral replication is ribavirin or is an interferon, such as an interferon-α; and in a particular embodiment, the interferon-α is pegylated. The invention also provides a method wherein castanospermine and the agent that alters viral replication are administered sequentially (wherein castanospermine is administered before the agent that alters viral replication or wherein the agent that alters viral replication is administered before castanospermine), or castanospermine and the agent are administered concurrently. In another embodiment, castanospermine and the agent that alters viral replication are admixed as a single composition and administered concurrently. In another embodiment, the method comprises administering to a subject (a) a composition comprising castanospermine and a pharmaceutically acceptable carrier and (b) a composition comprising the agent that alters viral replication and a pharmaceutically acceptable carrier.

The invention also provides a method for treating a Flaviviridae infection comprising administering to a subject castanospermine, or a pharmaceutically acceptable salt thereof, and an agent that alters immune function wherein castanospermine and the agent that alters immune function interact synergistically. In another embodiment, a method is provided for treating a Flaviviridae infection comprising administering to a subject castanospermine, or a pharmaceutically acceptable salt thereof, and an agent that alters viral replication wherein castanospermine and the agent that alters viral replication interact synergistically.

Also provided herein is a method for treating Flaviviridae infection comprising castanospermine in combination with an agent that inhibits infection of cells by Flaviviridae; a compound that inhibits the release of viral RNA from the viral capsid or inhibits the function of Flaviviridae gene products; a compound that alters Flaviviridae replication; a compound that alters immune function; a compound that alters symptoms of a Flaviviridae infection; or a compound for treating Flaviviridae-associated infections. In certain embodiments, the compound that alters immune function is an interferon such as an interferon-α; and in a particular embodiment, the interferon-α is pegylated. In a particular embodiment, the compound that alters Flaviviridae viral replication is ribavirin or interferon-α. In certain embodiments, the Flaviviridae-associated infection is a hepatitis B viral (HBV) infection or a retroviral infection, wherein the retroviral infection is a human immunodeficiency virus (HIV) infection.

Also provided is a method for treating a Flaviviridae infection, comprising administering to a subject castanospermine, or a pharmaceutically acceptable salt thereof, and an interferon. In certain embodiments, the interferon is an interferon-α, and in certain other embodiments, the interferon-α is pegylated. In one embodiment, castanospermine and the interferon interact synergistically.

### BRIEF DESCRIPTION OF THE DRAWINGS

FIGS. 1A and 1B show the 3-dimensional and 2-dimensional, respectively, synergy of interferon-α2b (IFN-α) and castanospermine (Cast) against MDBK cells that were infected with BVDV at a multiplicity of infection (MOI) of 0.01. The positive % above the horizontal plane (additivity surface) reveals the regions and corresponding drug concentrations at which a synergistic effect is observed. The gradation in gray, a shift from light to dark, indicates the level of synergy.

FIG. 2 shows an isobologram of the data shown in FIG. 1.

FIGS. 3A and 3B show the 3-dimensional and 2-dimensional, respectively, synergy of ribavirin (RBV) and castanospermine (Cast) against MDBK cells that were infected with BVDV at a multiplicity of infection (MOI) of 0.01. The positive % above the horizontal plane (additivity surface) reveals the regions and corresponding drug concentrations at which a synergistic effect is observed. The gradation in gray, a shift from light to dark, indicates the level of synergy.

FIG. 4 shows an isobologram of the data shown in FIG. 3.

FIGS. 5A and 5B show the 3-dimensional and 2-dimensional, respectively, results of a cytopathic assay in which MDBK cells were infected with BVDV at a multiplicity of infection (MOI) of 0.01 and then exposed to interferon-α2b (IFN-α) and castanospermine (Cast). The negative % below the horizontal plane (additivity surface) reveal the regions and corresponding drug concentrations at which cytotoxicity is not increased and even possibly diminished. The gradation in gray, a shift from light to dark, indicates the level of antagonism (i.e., unaffected cytotoxicity).

FIGS. 6A and 6B show the 3-dimensional and 2-dimensional, respectively, results of a cytopathic assay in which MDBK cells were infected with BVDV at a multiplicity of infection (MOI) of 0.01 and then exposed to ribavirin (RBV) and castanospermine (Cast). The negative % below the horizontal plane (additivity surface) reveal the regions and corresponding drug concentrations at which cytotoxicity is not increased and even possibly diminished. The gradation in gray, a shift from light to dark, indicates the level of antagonism (i.e., unaffected cytotoxicity).

### DETAILED DESCRIPTION

The present invention generally provides compositions and methods for using castanospermine in combination with another anti-viral compound to treat or prevent infectious diseases. In particular, these compositions are useful for treating or preventing Flaviviridae infections, such as hepatitis C virus (HCV) infections. The invention, therefore, relates generally to the surprising discovery that castanospermine in combination with another compound, such as interferon-alpha (IFN-α, interferon-α, alpha-interferon, or α-interferon) or ribavirin, has an unexpectedly high activity against HCV. Accordingly, the compounds of the invention are useful, for example, for treating HCV infection and HCV-related disease and are also useful as research tools for in vitro and cell-based assays to study the biological mechanisms of HCV infection (e.g., replication and transmission).

By way of background, glycoproteins are classified into two major classes according to the linkage between sugar and amino acid of the protein. The most common is the N-glycosidic linkage between an asparagine of the protein and an N-acetyl-D-glucosamine residue of an oligosaccharide. N-linked oligosaccharides, following attachment to a polypeptide backbone, are processed by a series of specific enzymes in the endoplasmic reticulum (ER), and this processing pathway has been well characterized.

In the ER, α-glucosidase I is responsible for the removal of the terminal α-1,2 glucose residue from the precursor oligosaccharide, and α-glucosidase II removes the two remaining α-1,3 linked glucose residues prior to removal of mannose residues by mannosidases and further processing reactions involving various transferases. These oligosaccharide “trimming” reactions enable glycoproteins to fold correctly and to interact with chaperone proteins such as calnexin and calreticulin for transport through the Golgi apparatus.

Inhibitors of key enzymes in this biosynthetic pathway, particularly those blocking α-glucosidases and α-mannosidases, prevent replication of several enveloped viruses. Such inhibitors may act by interfering with the folding of the viral envelope glycoprotein, thus preventing the initial virus-host cell interaction or subsequent fusion. These inhibitors may also prevent viral duplication by preventing the construction of the proper glycoprotein required for the completion of the viral membrane.

For example, nonspecific glycosylation inhibitors 2-deoxy-D-glucose and β-hydroxy-norvaline inhibited expression of HIV glycoproteins and blocked the formation of syncytia (Blough et al., Biochem. Biophys. Res. Commun. 141:33-38 (1986)). Viral multiplication of HIV-infected cells treated with these agents is stopped, presumably because of the unavailability of glycoprotein required for viral membrane formation. The glycosylation inhibitor 2-deoxy-2-fluoro-D-mannose exhibited antiviral activity against influenza-infected cells by preventing the glycosylation of viral membrane protein (McDowell et al., Biochemistry, 24:8145-52 (1985)). Lu et al. presented evidence that N-linked glycosylation was necessary for hepatitis B virus secretion (Virology 213: 660-665 (1995)), and Block et al. showed that secretion of human hepatitis B virus was inhibited by the imino sugar N-butyldeoxynojirimycin (Proc. Natl. Acad. Sci. USA 91: 2235-39(1994); see also, e.g., WO9929321).

As described herein, any concentration range, percentage range, ratio range or integer range is to be understood to include the value of any integer within the recited range and, when appropriate, fractions thereof (such as one tenth and one hundredth of an integer), unless otherwise indicated. As used herein, “about” or “comprising essentially of” mean ±15% of the indicated value or range, unless otherwise indicated. The use of the alternative (e.g., “or”) should be understood to mean either one, both, or any combination thereof of the alternatives.

Castanospermine

As set forth above, the present invention provides compositions having castanospermine, pharmaceutically acceptable salts thereof, and uses thereof. Disclosed herein are compositions having castanospermine in combination with another compound or molecule having anti-viral activity or castanospermine in combination with another compound or molecule that that alters host function or response, such as a compound that alters immune function or response, which combinations have unexpectedly high anti-viral activity, and in particular high anti-Flaviviridae activity, such as against HCV.

Castanospermine and certain imino sugars, such as deoxynojirimycin (DNJ), are ER α-glucosidase inhibitors, and both potently inhibit the early stages of glycoprotein processing (see, e.g, Ruprecht et al., J. Acquir. Immune Defic. Syndr. 2:149-57 (1989); see also, e.g., Whitby et al., Antiviral Chem. Chemother. (15:141-51 (2004); Branza-Nichita et al., J. Virol. 75:3527-36 (2001); Courageot et al., J. Virol. 75:564-72 (2000); Choukhi et al., J. Virol. 72:3851-58 (1998); WO 99/29321; WO 02/089780). However, the effects of the inhibitors differ substantially depending on the system to which they are applied, and they may exhibit quite different specificities, castanospermine being relatively specific for α-glucosidase I.

Castanospermine is a natural alkaloid derived from the black bean or Moreton chestnut tree (Castanospermum australe) (Hohenschutz et al., Phytochemistry 20:811-14 (1981)). Castanospermine is water soluble and thus is readily isolated according to procedures practiced in the art (see, e.g., Alexis Platform, San Diego, Calif.). The highest concentration of the compound is found in the seeds and seed pods (Pan et al., Arch. Biochem. Biophys. 303:134-44 (1993)). In addition to inhibiting the enzymatic activity of α-glucosidase I, castanospermine also inhibits intestinal glycosidases, such as maltase and sucrase, which may result in undesirable side effects (Saul et al., Proc. Natl. Acad. Sci. USA 82:93-97 (1985)). Many side effects may be minimized or prevented in a subject receiving castanospermine by altering the subject's diet to a starch-free, high-glucose diet (see, e.g., Saul et al., supra).

Castanospermine has the following formula, 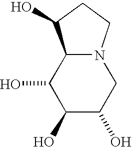
Systematically, this compound can be named in several ways: [1S-(1α,6β,7α,8β,8αβ)]-octahydro-1,6,7,8-indoli-zinetetrol or [1S-(1S,6S,7R,8R,8aR)-1,6,7,8-tetrahydroxy-indolizidine or 1,2,4,8-tetradeoxy-1,4,8-nitrilo-L-glycero-D-galacto-octitol. The term castanospermine or the first systematic name will be used herein.

Pharmaceutically acceptable salt refers to a salt of castanospermine or other compounds described herein that is pharmaceutically acceptable and that possesses the desired pharmacological (e.g., anti-viral) activity. Such salts include the following: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine, and the like.

A structurally pure compound refers to a compound composition in which a substantial percentage, e.g., on the order of 95% to 100% and preferably ranging from about 95%, 96%, 97%, 98%, 99% or greater, of the individual molecules comprising the composition each contain the same number and types of atoms attached to each other in the same order and with the same bonds. As used herein, the term “structurally pure” is not intended to distinguish different geometric isomers or different optical isomers from one another. For example, a mixture of cis- and trans-but-2,3-ene is considered structurally pure, as is a racemic mixture. When compositions are intended to include a substantial percentage of a single geometric isomer or optical isomer, the terms “geometrically pure” and “optically or enantiomerically pure,” respectively, are used.

The term “structurally pure” is also not intended to discriminate between different tautomeric forms or ionization states of a molecule, or other forms of a molecule that result from equilibrium phenomena or other reversible interconversions. Thus, a composition of, for example, an organic acid is structurally pure even though some of the carboxyl groups may be in a protonated state (COOH) and others may be in a deprotonated state (COO⁻). Likewise, a composition comprising a mixture of keto and enol tautomers, unless specifically noted otherwise, is considered structurally pure.

Therapeutic Formulations and Methods of Use

As described herein, castanospermine, particularly in combination with another agent (compound or molecule), such as an agent that alters immune function and/or an agent that alters viral replication, may act synergistically to inhibit viral infection or viral replication. In certain embodiments, the combinations described herein are capable of inhibiting viral replication of a virus of the Flaviviridae family, preferably HCV, at clinically relevant concentrations according to statistically measurable criteria. Use of castanospermine in combination with at least one other therapeutic agent described herein as a treatment encompasses therapeutic application, that is, administration of the combination to a subject known to be or believed to be infected with a virus of the Flaviviridae family, such as HCV. Also contemplated herein, is a combination of castanospermine with an agent, such as interferon-α, wherein the combination with castanospermine may alter (increase or decrease, preferably increase, in a statistically significant manner) the effectiveness (efficacy) of interferon-α or ribavirin for treating a Flaviviridae infection.

Treatment also encompasses prophylaxis or preventative administration of the combinations described herein. Effective treatment of a Flaviviridae infection may include a cure of the infection (i.e., eradication of the virus from the host or host tissue); a sustained response in which, for example, HCV RNA is not longer detectable in the blood of the subject six months after completing a therapeutic regimen (such a sustained response may be equated with a favorable prognosis and may be equivalent to a cure); slowing or reducing liver scarring (fibrosis); slowing or reducing the production of virus; reducing, alleviating, or abrogating symptoms in a subject; or preventing symptoms or infection from worsening or progressing.

Thus, the compositions described herein may be used for accomplishing at least one of the following goals: (1) elimination of infectivity and potential transmission of a Flaviviridae infection, such as an HCV infection, to another subject; (2) arresting the progression of liver disease and improving clinical prognosis; (3) preventing development of cirrhosis and HCC; and (4) improving the clinical benefit of currently used therapeutic molecules or modalities. To date, a therapeutic agent that adequately treats or prevents an HCV infection and any associated disease without severe side-effects has remained elusive.

The therapy or prophylaxis is preferably the treatment or prevention of an infection by a virus as defined above. In particular, the therapy or prophylaxis may be the treatment or prevention of a disease selected from hepatitis C, yellow fever, dengue fever, Japanese encephalitis, Murray Valley encephalitis, Rocio virus infection, West Nile fever, St. Louis encephalitis, tick-borne encephalitis, Louping ill virus infection, Powassan virus infection, Omsk hemorrhagic fever, Kyasanur forest disease, bovine diarrhea, classical swine fever, border disease, and hog cholera. A viral infection, such as a Flaviviridae infection (e.g., an HCV infection), refers to any state or condition that involves (i.e., is caused, exacerbated, or characterized by) a flavivirus residing in the cells or body of the subject or patient. A patient or subject may be a human, a non-human primate, sheep, cattle, horse, pig, dog, cat, rat, mouse, or other mammal.

HCV is difficult to propagate efficiently in cell culture, thus rendering analysis and identification of potential anti-HCV agents difficult. In the absence of a suitable cell culture system capable of supporting replication of human HCV and re-infection of cells in vitro, use of another member of the Flaviviridae family, bovine viral diarrhea virus (BVDV) is an art-accepted surrogate virus for use in cell culture models (Stuyver et al., Antimicrob. Agents Chemother. 47:244-54 (2003); Whitby et al., supra). HCV and BVDV share a significant degree of local protein homology, a common replication strategy, and probably the same subcellular location for viral envelopment. Both HCV and BVDV have single-stranded genomes (approximately 9,600 and 12,6000 nucleotides, respectively) that encode nine functionally analogous gene products, including the E1 and E2 envelope glycoproteins (see, e.g., Rice, Flaviviridae: The Viruses and Their Replication, in Fields Virology, 3rd Ed. Philadelphia, Lippincott, 931-59 (1996)).

The compounds described herein may also be useful research tools for in vitro and cell-based assays to study the biological mechanisms of viral infection, growth, and replication, preferably for HCV. By way of background and not wishing to be bound by theory, HCV morphogenesis is complex wherein preassembled viral core particles are believed to attach to cytosolic sides of viral envelope (surface) proteins, which have inserted in the endoplasmic reticulum (ER) membrane. After acquiring envelopes, virions bud to the lumen of the ER and then are transported through the Golgi apparatus to the extracellular fluids. Removal of N-linked glucose residues (trimming is done by cellular enzymes, such as α-glucosidases) from immature viral glycoproteins may play a role in the migration of viral glycoproteins from the ER to the Golgi.

In one embodiment, a method is provided for identifying anti-viral compounds, comprising contacting a host cell infected with a virus with castanospermine and one other test compound or agent under conditions and for a time sufficient to inhibit viral replication, and identifying a candidate agent that inhibits (prevents, slows, abrogates, interferes with) infection, viral replication, and/or viral assembly. In certain embodiments, the methods described herein may be used to identify a test compound that acts additively or synergistically when combined with castanospermine. In another embodiment, a method is provided for identifying cells suspected of having a viral infection, comprising contacting a host cell suspected of being infected with a virus with castanospermine and one candidate compound or agent under conditions and for a time sufficient to inhibit infection, viral replication, and/or viral assembly, and identifying cells infected with a virus. Preferably, the viral infection is caused by or associated with HCV. The assays described herein may be used to determine the therapeutic value of a candidate compound and/or combination and also may be useful for determining dosage parameters that would be useful in treating a subject in need thereof.

In particular embodiments, castanospermine is administered in association or in combination (e.g., in an admixture or co-packaged or administered in such a manner that castanospermine and the one other compound are available systemically or at the site of infection, such that the anti-viral effects of each may be additive or preferably synergistic) when combined with an adjunctive therapeutic. In one preferred embodiment, castanospermine is combined with an agent that alters immune function, such as interferon-α, and in another preferred embodiment, castanospermine is combined with an agent that alters viral (e.g., Flaviviridae) replication, for example, a nucleoside analog, such as ribavirin (Sigma). In another embodiment, castanospermine is combined or administered in association with an agent that alters an immune function, such as interferon-α, or an agent (compound) that alters viral replication, such as ribavirin.

In one embodiment, the combination of castanospermine and an agent that alters immune function or the combination of castanospermine and an agent that alters viral replication act additively in the subject. That is, the interaction of castanospermine and another compound result in a therapeutic effect (or anti-viral effect) that is approximately equal to the effect of each compound or agent if it were administered alone.

A compound or molecule that has anti-viral activity may, for example, inhibit or prevent infection of a cell (such as by preventing binding or adherence of the virus to a cell); inhibit, reduce, or prevent viral replication or assembly; inhibit, reduce, or prevent release of viral RNA from the viral capsid; and/or inhibit, reduce, or interfere with the function of a HCV gene product. A compound or molecule that alters immune function (increases or decreases in a statistically significant manner or a clinically significant manner) preferably increases or enhances an immune function or immune response against the infectious virus.

In a preferred embodiment, the combination of castanospermine and an agent that alters immune function or the combination of castanospermine and an agent that alters viral replication act synergistically in the subject. Two or more compounds that act synergistically interact such that the combined effect of the compounds is greater than the sum of the individual effects of each compound when administered alone (see, e.g., Ouzounov et al., Antivir. Res. 55:425-35 (2002); Berenbaum, Pharmacol. Rev. 41:93 (1989)). An interaction between castanospermine and another agent or compound may be analyzed by a variety of mechanistic and empirical models (see, e.g., Ouzounov et al., supra). A commonly used approach for analyzing interaction between a combination of agents employs the construction of isoboles (iso-effect curves), in which the combination of agents (dₐ,d_{b}) is respresented by a point on a graph, the axes of which are the dose-axes of the individual agents (see, e.g., Ouzounov et al., supra; MacSynergy™ II software manual (University of Michigan, Ann Arbor, Mich.). Castanospermine in combination with an agent that alters immune function or castanospermine in combination with an agent that alters viral replication or another agent or compound described herein act synergistically or have a synergistic effect when the volume of synergy produced as calculated by the volume of the synergy peaks is 15% greater than the additive effect (that is, the effect of each agent alone added together), or that is 2-fold greater than the additive effect, or that is 3- or more fold greater than the additive effect. Synergy may be described using the 3-dimensional (3-D) graphs and synergistic volume calculations provided by the MacSynergy™ II software as a complementary analysis to isobolograms. Accordingly, castanospermine in combination with an agent that alters viral replication or castanospermine in combination with an agent that alters immune function, or another agent or compound described herein, act synergistically or have a synergistic effect when values expressed in μM/ml²% or μM(IU)/ml(well)²% are between 25 and 50 μM/ml²% or 25 and 50 μM(IU)/ml(well)²% (minor but statistically significant); between 50 and 100 μM/ml²% or μM(IU)/ml(well)²% (moderate synergy); or greater than 100 μM/ml²% or μM(IU)/ml(well)²% (strong synergy). Buckwold et al. reported that ribavirin and interferon-α in combination (which drug combination is the current standard of care for treating HCV infections) had a synergy volume of 66±25 IU(μg)/ml(well)²% (Antimicrob. Agents Chemother. 47:2293 (2003)). A combination of castanospermine and interferon-α as described herein had a synergy volume ranging from 193 to 244 μM/(IU/ml)²%.

In certain embodiments, there is provided a composition comprising castanospermine in combination with a compound that inhibits the binding to and/or infection of cells by Flaviviridae, such as HCV. Examples of such compounds include antibodies that specifically bind to one or more Flaviviridae gene products (e.g., HCV E1 and/or E2 proteins) or to a cell receptor to which the HCV binds. The antibody may be a monoclonal or polyclonal antibody, or antigen binding fragments thereof, including genetically engineered chimeric, humanized, sFv, or other such immunoglobulins. Other compounds that prevent binding or infection of cells by a virus include glucosaminoglycans (such as heparan sulfate and suramin).

Castanospermine may also be combined with a compound that inhibits the release of viral RNA from the viral capsid or inhibits the function of Flaviviridae, such as HCV, gene products, including inhibitors of the IRES, serine protease inhibitors, helicase inhibitors, and inhibitors of viral polymerase/replicase (see, e.g., Olsen et al., Antimicrob. Agents Chemother. 48:3944-53 (2004); Stansfield et al., Bioorg. Med. Chem. Lett. 14:5085-88 (2004)). Inhibitors of IRES include, for example, nucleotide sequence specific antisense (see, e.g., McCaffrey et al., Hepatology 38:503-508 (2003)); small yeast RNA (see, e.g., Liang et al., World J. Gastroenterol. 9:1008-13 (2003)); or short interfering RNA molecules (siRNA) that inhibit translation of mRNA; and cyanocobalamin (CNCb1, vitamin B12) (Takyar et al., J. Mol. Biol. 319:1-8 (2002)). NS3 protease (helicase) inhibitors include peptides that are derived from NS3 substrates and act to block enzyme activity. Protease inhibitors designated BILN 2061 (see, e.g., Lamarre et al., Nature 426:186-89 (2003) (Boehringer Ingelheim Pharma, Quebec) and VX-905 (Vertex Pharmaceuticals, Inc. Cambridge, Mass.) have been investigated as potential HCV therapeutics.

In another embodiment, castanospermine may be combined with a compound that perturbs cellular functions involved in or influencing Flaviviridae replication, or that directly alters Flaviviridae replication, including inhibitors of RNA-dependent RNA polymerase or inhibitors of HCV p7 (e.g., DGJ and derivatives), other inhibitors of glycoprotein processing (such as imino sugars, including deoxygalactonojirimycin (DGJ) and deoxynojirimycin (DNJ), and derivatives thereof (e.g., N-butyl-DNJ, N-nonyl-DNJ, and long alkyl chain imino sugars such as N7-oxanonyl-DNJ, N7-oxanonyl-DGJ)), nucleoside analogues including inhibitors of inosine monophosphate dehydrogenase (e.g., ribavirin, mycophenolic acid, and VX497), and other antiviral compounds such as amantadine, (Symmetrel®, Endo Pharamceuticals), rimantadine (Flumadine®, Forest Pharmaceuticals, Inc.), valopicitabine (NM283, Idenix Pharmaceuticals).

In another embodiment, castanospermine may be combined with a compound that acts to alter immune function (increase or decrease in a statistically significant, clinically significant, or biologically significant manner), preferably to enhance or stimulate an immune function or an immune response against a Flaviviridae infection. For example, a compound may stimulate a T cell response or enhance a specific immune response (e.g., thymosin-α, and interferons such as α-interferons and β-interferons) or may stimulate or enhance a humoral response. Examples of compounds that alter an immune function include type I interferons, such as interferon-α (see, e.g., Nagata et al., Nature 287:401-408 (1980)), interferon-β (see, e.g, Tanigushi et al., Nature 285:547-49 (1980)), and interferon-ω (Adolf, J. Gen. Virol. 68:1669-76 (1987)), and type II interferons, such as interferon-γ (Belardelli, APMIS 103:161, 1995) and interferon-γ-1b (Alferon). Examples of interferon-α include interferon-α-2a (Roferon®-A; Hoffman-La Roche), interferon-α-2b (Intron A, PBL Biomedical), interferon-α-con-1 (Infergen), interferon-α-n3 (Alferon), albumin interferon-α (Albuferon-alpha™, Human Genome Sciences, Rockville, Md.) and Veldona (Amarillo Biosciences, Inc.). Examples of interferon-β, include interferon-β-1a (Avonex) and interferon-β-1b (Betaseron).

Interferons may alter immune function and also may alter (inhibit, prevent, abrogate, reduce, or slow) replication of a virus, such as HCV. The production of interferon-α and interferon-β in virally infected cells induces resistance to viral replication, enhances MHC class I expression, increases antigen presentation, and activates natural killer cells (subset of lymphocytes that lack antigen-specific surface receptors) to kill virus-infected cells (see, e.g., Janeway et al., in Immunobiology, 5th ed. New York, London: Garland Publishing, (2001)). Thus, these interferons alter immune function by affecting both innate and adaptive immunity.

In one embodiment, castanospermine is administered in combination with the interferon, such as interferon-α. Interferon-α has been used in the treatment of a variety of viral infections, either as a monotherapy or as part of a combination therapy (see, e.g, Liang, New Engl. J. Med. 339:1549-50 (1998); Hulton et al., J. Acquir. Immune Defic. Syndr. 5:1084-90 (1992); Johnson et al., J. Infect. Dis. 161:1059-67 (1990)). Interferon-α binds to cell surface receptors and stimulates signal transduction pathways that lead to activation of cellular enzymes (e.g., double-stranded RNA-activated protein kinase and RNase L that inhibit translation initiation and degrade viral RNA, respectively) that repress virus replication (see, e.g., Samuel, Clin. Microbiol. Rev. 14:778-809 (2001); Kaufman, Proc. Natl. Acad. Sci. USA 96:11693-95 (1999)). HCV E2 glycoprotein and NS5a may block RNA-activated protein kinase activity such that some HCV strains are more resistant to interferon-α; thus, combination therapies of interferon-α and one or more other compounds may be necessary for treatment of persistent viral infection (see, e.g., Ouzounov et al., supra, and references cited therein). In a certain embodiment, a polyethylene glycol moiety is linked to interferon-α (known as pegylated interferon-α; peginterferon α-2b (Peg-Intron®; Schering-Plough or PBL Biomedical) and peginterferon α-2a (Pegasys®; Hoffmann-La Roche), which may have an improved pharmacokinetic profile and may also manifest fewer undesirable side effects (see, e.g., Zeuzem et al., New Engl. J. Med. 343:1666-72 (2000); Heathcote et al., New Engl. J. Med. 343:1673-80 (2000); Matthews et al., Clin. Ther. 26:991-1025 (2004)).

Interferon-α-2a (Roferon®-A; Hoffman-La Roche), Interferon-α-2b (Intron®-A; Schering-Plough), and interferon-αcon-1 (Infergen®; Intermune) are approved for use as single agents in the U.S. for treatment of adults with chronic hepatitis C infection. The recommended dose of interferons-α-2b and -α-2a for the treatment of chronic hepatitis C infection is 3,000,000 units three times a week, and administered by subcutaneous or intramuscular injection. Treatment is administered for six months to two years. For interferon-α-con-1, the recommended dose is 9 μg three times a week for first time treatment and 15 μg three times a week for another six months for patients who do not respond or relapse. During the treatment periods with any of these recombinant interferons, the patient must be monitored for side effects, which include flu-like symptoms, depression, rashes, and abnormal blood counts. Treatment with interferon alone leads to a sustained response in less than 15% of subjects. Because of this low response rate, these interferons are rarely used as a monotherapy for the treatment of patients with chronic hepatitis C infection.

The combination of an interferon-α with ribavirin for treating an HCV infection has been superior to either treatment alone, and the combination is the current standard of care. The effectiveness, doses, and frequency of administration were studied in three large double-blind, placebo-controlled clinical trials (Reichard et al., Lancet 351:83-87 (1998); Poynard et al., Lancet 352:1426-32 (1998); McHutchison et al., New Engl. J. Med. 339:1485-92 (1998)). (See also Buckwold et al., Antimicrob. Agents Chemother. 47:2293-93 (2003); Buckhold, Antimicrob. Chemother. 53:412-14 (2004)). Adverse effects associated with ribavirin include abnormal fetal development. Ribavirin is also contraindicated in patients who have anemia, heart disease, or kidney disease. Therefore, alternative compositions, therapies, and therapeutic combinations such as those described herein are needed.

Castanospermine may also be combined with a compound that modulates (preferably decreases or reduces the severity or intensity of, reduces the number of, or abrogates) the symptoms and effects of a Flaviviridae infection, such as an HCV infection (e.g., antioxidants such as the flavinoids).

The adjunctive therapeutic may comprise an anti-viral compound, for example, an anti-viral compound or drug that is used for treatment of an infectious agent frequently identified as co-infecting a subject who is infected with a Flaviviridae, such as HCV. Such co-infection may be caused by HBV, a human retrovirus such as HIV1 and 2, and/or human T-cell lymphotrophic virus (HTLV) type 1 or type 2. Examples of anti-viral compounds include, but are not limited to, nucleotide reverse transcriptase (RT) inhibitors (e.g., Lamivudine (3TC), zidovudine, stavudine, didanosine, adefovir dipivoxil, and abacavir); non-nucleoside RT inhibitors (e.g., nevirapine); and aspartyl protease inhibitors (e.g., saquinavir, indinavir, and ritonavir).

The adjunctive therapeutics discussed herein may be administered concurrently as a single composition in a pharmaceutically acceptable carrier, or concurrently as separate compositions each having a pharmaceutically acceptable carrier. In another embodiment, the castanospermine and adjunctive therapeutic can be sequentially administered, or in any combination thereof (e.g., castanospermine first and adjunctive therapy second, or adjunctive therapy first and castanospermine second). Furthermore, if more than one dose of the combination therapy is administered, the order of the sequential administration can be changed or kept the same at each time point of administration. Methods for determining the effects of any combination therapy described herein may be determined by methods described herein and routinely practiced by a skilled artisan, such as determining whether an immune response has been altered, determining whether symptoms or effects of a Flaviviridae infection have been modulated, or determining whether Flaviviridae replication has been altered (preferably adversely affected, prevented, decreased, inhibited or abrogated viral replication).

As described herein, BVDV is an art-accepted surrogate virus for use in cell culture models (Stuyver et al., supra; Whitby et al., supra). Assays may therefore be performed using bovine cell lines, such as bovine kidney cells (MDBK) and bovine turbinate (BT) cells, using a cytopathic strain of BVDV such as the NADL strain (available from ATCC, Manassas, Va.) that causes cytolysis of infected cells. Exemplary assays that may be performed to determine whether castanospermine alone or in combination with another compound, agent, or molecule may be useful for treating a Flaviviridae infection or inhibiting or preventing a Flaviviridae infection include viral plaque formation assays, cytotoxicity assays (see, e.g., Buckwold et al., Antimicrob. Agents Chemother. 47:2293-98 (2003); Whitby et al., supra), virus release assays, cell proliferation assays (e.g., nonradioactive MTS/PMS or MTT assays, or radioactive thymidine incorporation assays), and other assays described herein and known and practiced by persons skilled in the art. The data from these assays when castanospermine are analyzed in combination with another compound, such as data obtained from the cytotoxicity assay, may be analyzed as described herein to determine whether the agents interact to provide an additive effect or a synergistic effect.

The invention also relates to pharmaceutical compositions that contain castanospermine in combination with another compound used to treat or prevent a viral infection (e.g., HCV). The invention further relates to methods for treating or preventing viral infections by administering to a subject castanospermine in combination with one other compound, wherein each component is administered at a dose sufficient to treat or prevent a viral infection, as described herein. The castanospermine and combinations or cocktails of such compounds, are preferably part of a pharmaceutical composition when used in the methods described herein. Castanospermine may be administered in combination with another compound described herein by administering each compound sequentially to a subject, that is, castanospermine may be administered prior to administration of another compound, after administration of another compound; alternatively castanospermine may be administered concurrently with another compound. For sequential or concurrent administration of each compound (molecule, agent) of a combination described herein, each compound may be administered by the same or different routes in the same or different formulations, which are described herein and determined, in part, according to the properties of the compounds.

In one embodiment, the invention comprises a pharmaceutical composition comprising castanospermine as described herein (or a pharmaceutical salt thereof) and a pharmaceutically acceptable carrier, vehicle, or excipient, and optional additives (e.g., one or more binders, colorings, desiccants, stabilizers, diluents, or preservatives) for use in the methods of treatment described herein. Similarly, an adjunctive therapy, such as interferon-α or ribavirin, may be combined with a pharmaceutically acceptable carrier, vehicle, or excipient, and optional additives. Pharmaceutical compositions comprising interferon-α or ribavirin may be prepared according to methods known and practiced in the art for preparing these compounds for administration to a subject.

As set forth herein, castanospermine and one more adjunctive therapeutic compound or molecule may be included in a pharmaceutically acceptable carrier, excipient, or diluent for administration to a subject in need thereof in an amount effective to treat or prevent a Flaviviridae infection, particularly, an HCV infection. In certain embodiments, a dose of the active compound(s) for all indications described herein is administered in a range from about 0.01 mg/kg to about 300 mg/kg per day, preferably about 0.1 mg/kg to about 100 mg/kg per day, or more preferably about 0.5 mg/kg to about 25 mg/kg body weight of the recipient per day. A topically administered dosage can range from about 0.01-3% wt/wt in a suitable carrier. Interferon-α or ribavirin when administered in combination with castanospermine may be administered according to dosing regimens known and practiced in the art (see, e.g., Matthews et al., supra; Foster, Semin. Liver Dis. 24 Suppl 2:97-104 (2004); Craxi et al., Semin Liver Dis. 23 Suppl 1:35-46 (2003)), or the dosing may be adjusted when administered with castanospermine.

The active ingredient(s) are preferably administered to achieve peak plasma concentrations of about 0.001 μM to about 30 μM, and preferably about 0.01 μM to about 10 μM. This may be achieved, for example, by intravenous injection of a composition of a formulation of castanospermine, optionally in saline or other aqueous medium. In another embodiment, castanospermine is administered as a bolus. Castanospermine and other compounds used in the methods of treatment described herein may be administered orally, or intramuscularly, intraperitoneally, subcutaneously, transdermally, via an aerosol or by inhalation, rectally, vaginally, or topically (including buccal and sublingual administration).

The concentration of an active compound in a pharmaceutical composition will depend on absorption, distribution, inactivation, and excretion rates of the compound, as well as other factors known to those of skill in the art. The dose will also vary with the severity of the condition to be alleviated. Specific dose regimens (including frequency of dose administration) may be adjusted over time according to the individual subject's need and the professional judgment of the person administering or supervising the administration of the compositions. The dose level and regimen will depend on a variety of factors, including the age, body weight, diet, gender, general health, and medical history (including whether the subject is co-infected with another virus, such as HBV or HIV). Accordingly, the concentration ranges set forth herein are exemplary only and are not intended to limit the scope or practice of the claimed compositions. The active ingredient may be administered at once, or may be divided into a number of smaller doses to be administered at varying intervals of time.

The compositions for pharmaceutical use as described herein may be in the form of a kit of parts. The kit may comprise, for example, castanospermine, as one component of the composition in unit dosage form, and may also comprise an agent that alters immune function (e.g., interferon-α) or an agent that alters viral replication (such as ribavirin), each in the respective dosage unit form. The kit may include instructions for use and other relevant information, as well as information required by a regulatory agency.

Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a dispersing agent such as alginic acid, Primogel, or corn starch; a lubricant such as magnesium stearate or Sterores; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials that modify the physical form of the dosage unit, for example, coatings of sugar, shellac, or enteric agents. See generally “Remington's Pharmaceutical Sciences,” Mack Publishing Co., Easton, Pa.

The active compound or pharmaceutically acceptable salt or derivative thereof can be administered as a component of an elixir, suspension, syrup, wafer, chewing gum or the like. A syrup may contain, in addition to the active compounds, sucrose as a sweetening agent and certain preservatives, dyes and colorings, and flavors.

The pharmaceutical composition described herein will preferably include at least one of a pharmaceutically acceptable vehicle, carrier, diluent, or excipient, in addition to castanospermine, and other components or active ingredients (such as other anti-HBV drug), including agents that alter viral replication or alter an immune function or response, and/or an agent that is an anti-Hepadnaviridae (e.g., anti-HBV), which are described in detail herein. A composition of the invention may have a variety of active ingredients, such as castanospermine or pharmaceutically acceptable salt thereof, or a cocktail or combination of with one or more antibiotics, antifungals, anti-inflammatory agents, or other anti-viral compound.

Pharmaceutically acceptable carriers suitable for use with a composition may include, for example, a thickening agent, a buffering agent, a solvent, a humectant, a preservative, a chelating agent, an adjuvant, and the like, and combinations thereof. Pharmaceutically acceptable carriers for therapeutic use are well known in the pharmaceutical art, and as described herein and, for example, in Remington's Pharmaceutical Sciences, Mack Publishing Co. (A. R. Gennaro, ed., 18^{th} Edition, 1990) and in CRC Handbook of Food, Drug, and Cosmetic Excipients, CRC Press LLC (S. C. Smolinski, ed., 1992).

Solutions or suspensions used for parenteral, intradermal, subcutaneous, or topical application can include the following components: a sterile diluent such as water for injection, saline solution, fixed oils, polyethylene glycols, glycerin, propylene glycol or other synthetic solvents; anti-bacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes, or multiple dose vials made of glass or plastic. If administered intravenously, preferred carriers are physiological saline or phosphate buffered saline (PBS) or an adjuvant. Exemplary adjuvants are alum (aluminum hydroxide, REHYDRAGEL®); aluminum phosphate; virosomes, liposomes with and without Lipid A, Detox (Ribi/Corixa); MF59; or other oil and water emulsions type adjuvants, such as nanoemulsions (see, e.g., U.S. Pat. No. 5,716,637) and submicron emulsions (see, e.g., U.S. Pat. No. 5,961,970), and Freund's complete and incomplete. In preferred embodiments, a pharmaceutical composition is sterile.

In certain embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. For example, as is known in the art, some of these materials can be obtained commercially from Alza Corporation (CA) and Gilford Pharmaceuticals (Baltimore, Md.).

Liposomal suspensions may also be pharmaceutically acceptable carriers. These may be prepared according to methods known to those skilled in the art (for example, U.S. Pat. No. 4,522,811; U.S. Pat. No. 6,320,017; U.S. Pat. No. 5,595,756). For example, liposome formulations may be prepared by dissolving appropriate lipid(s) (such as stearoyl phosphatidyl ethanolamine, stearoyl phosphatidyicholine, arachadoyl phosphatidylcholine, and cholesterol) in an inorganic solvent that is then evaporated, leaving behind a thin film of dried lipid on the surface of the container. An aqueous solution of the active compound or its monophosphate, diphosphate, and/or triphosphate derivatives are then introduced into the container. The container is then swirled by hand to free lipid material from the sides of the container and to disperse lipid aggregates, thereby forming the liposomal suspension. Hydrophilic compounds, such as castanospermine, may likely be loaded into the aqueous interior of a liposome.

All U.S. patents, U.S. patent application publications, U.S. patent applications, foreign patents, foreign patent applications, and non-patent publications referred to in this specification, are incorporated herein by reference, in their entirety. The following examples are intended to illustrate, but not limit, the invention.

### EXAMPLES

### Example 1

### Protection of MDBK Cells from BVDV-Induced Cytotoxicity by Castanospermine, Interferon-α2b and Ribavirin

Cell proliferation assays were performed using a non-radioactive cell proliferation MTS/PMS assay (MTS: 3-(4,5-dimethylthiazol-2-yl)-5-(3-carboxymethoxy phenyl)-2-(4-sulfophenyl)-2H-tetrazolium (Promega Corporation, Madison, Wis.); PMS: phenazine methosulfate (Sigma Aldrich, St. Louis, Mo.)). MDBK cells were seeded into 96-well plates at a density of approximately 2×10⁴ cells per well. The cultures were incubated 3-24 hours to permit attachment of the cells to the plates prior to infection and addition of compounds. The appropriate number of PFU of BVDV were added to each well to achieve the desired MOI (0.1 or 0.01); the cells were exposed to the virus diluted at the appropriate concentration in phosphate buffered saline (PBS) containing 1% horse serum for 1 to 2 hours. The virus inoculum was then removed and the cells were washed with PBS containing 1% HS. The test compounds, castanospermine, ribavirin, and interferon-α were dissolved in cell growth media with 2% HS and added to the cells at varying concentrations, and then incubated at 37° C. in the presence of 5% CO₂ for 3-4 days. Uninfected cells and infected, untreated cells (i.e., without compound) were used as additional controls. After the 3-4 days of incubation, a combined MTS/PMS solution was added into each well assay plate containing 100 μL of cells in culture medium to obtain final concentrations of 333 μg/ml MTS and 25 μM PMS. A spectrophotometer plate reader was used to measure the absorbance at 490 nm after incubation of the 96-well plate for 1 to 4 hours at 37° C. in a humidified, 5% CO₂ atmosphere. The mean absorbance in each set of triplicate wells was determined. Antiviral activity was measured as MTS conversion relative to the differential between the conversion for cell (non-infected) and viral (non-drug-treated) controls. The cytopathic effect (CPE) reduction for each concentration of the tested compound, which correlated with antiviral activity, was calculated as follows. % CPE reduction=[(D−ND)/(NI−ND)]×100, in which D (drug-treated) is the absorbance of drug-treated cells; ND (non drug-treated) is the absorbance of untreated infected cells; and NI (non-infected) is the absorbance of non-infected cells. The data are presented in Table 1.

TABLE 1
Protection of MDBK Cells from BVDV-Induced Cytotoxicity

| | MOI = 0.01 | | MOI = 0.1 | | | |
|---|---|---|---|---|---|---|
| Compound | EC₅₀ | | EC₅₀ | | CC₅₀ | |
| Castanospermine | 61.5 ± 15.4 | μM | 177 ± 14.5 | μM | >1000 | μM |
| Interferon-α2b | 19.4 ± 6 | IU/well | 96.5 ± 22 | IU/well | >300 | IU/well |
| Ribavirin | 4.4 ± 2 | μM | 9 ± 1 | μM | 57 ± 34 | μM |

*IU = interferon units

The EC₅₀ represents the concentration of the drug that protects 50% of the cells from BVDV induced cytotoxicity (i.e., 50% CPE reduction). CC₅₀ equals the concentration that affects the viability of 50% of the MDBK cells. These results indicate that each of the tested compounds have an anti-viral effect, which may be direct or indirect. Examples 2 and 3 disclose that the anti-viral effect is actually due to a direct effect on the virus.

### Example 2

### In Vitro Inhibition of BVDV Release from MDBK Cells By Castanospermine, Interferon-α2B and Ribavirin

Madin-Darby Bovine Kidney Cells (MDBK) (American Type Culture Collection (ATCC), Manassas, Va.; ATCC CCL22) were seeded into 96-well plates at a density of approximately 2×10⁴ cells per well in growth medium (e.g., Dulbecco's Modified Eagles Medium (DMEM); Gibco, Ontario, Canada) containing 2% heat inactivated horse serum (HS). The cell cultures were incubated 3-24 hours to allow attachment of the cells to the tissue culture plates prior to infection and addition of compounds. The appropriate number of plaque forming units (PFU) of BVDV strain NADL (ATCC VR-534) diluted in sterile PBS containing 1% HS were added to each well to achieve the desired multiplicity of infection (MOI) (about 0.01). The cells were exposed to the virus at 37° C., 5% CO₂ for 1.5 hours, and then washed with PBS. The test compounds, dissolved in cell growth media with 2% HS, were then added to the cells at varying concentrations. The plates were incubated at 37° C., 5% CO₂ for 24 hours (one cycle of BVDV replication). The 96-well plates were then centrifuged at low speed to pellet the cells. The supernatant was serially diluted and used to infect a new monolayer of cells in 12-well plates. The cell monolayer was then overlaid with 0.5% agarose dissolved in cell growth media with 5% HS containing (1) castanospermine (Phytex, Australia); (2) ribavirin (Ribavirin, Sigma); (3) IFN-α2b (PBL Biomedical Laboratories, Piscataway, N.J.); or (4) alone (i.e., no added test compound). The treated cells were incubated for 3 to 5 days at 37° C. under 5% CO₂, fixed with formaldehyde, stained with crystal violet or methylene blue, washed in double distilled water, and finally air dried at room temperature. Virus plaques formed were quantified by manual counting, and titer was determined. The data are presented in Table 2.

TABLE 2
Inhibition of Viral Release

| Compound | EC₅₀ | | EC₉₀ | |
|---|---|---|---|---|
| Castanospermine | 14 ± 8 | μM | 83 ± 21 | μM |
| Interferon-α2b | 0.4 | IU/well* | 1.4 ± 0.4 | IU/well* |
| Ribavirin | 2.4 ± 2.2 | μM | 4.9 ± 1.9 | μM |

*IU = interferon units

The EC₅₀ is the concentration of compound that inhibits 50% of viral release in the media of infected cells compared to the untreated control. These results support the data found in Example 1 and further demonstrate that each of the tested compounds have direct anti-viral effect, which indicates that HCV would also be directly inhibited by castanospermine, interferon and ribavirin.

### Example 3

### Plaque Inhibition Assay

The BVDV virus stock was serially diluted in phosphate buffered saline (PBS) containing 1-5% horse serum (HS). MDBK cells were grown to confluence in culture dishes and were infected at 37° C. with BVDV at various multiplicity of infection (<1 to >0.001). After 1.5 hours of adsorption, the inoculum was removed. The cell monolayer was overlaid with 0.5% agarose dissolved in cell growth media containing 5% HS with and without test compound(s) (castanospermine, ribavirin, or IFN-α). The dishes were incubated for 3 to 5 days at 37° C. under 5% CO₂. The monolayer of MDBK cells was fixed with formaldehyde, stained with crystal violet or methylene blue according to standard methods, and then washed in double distilled water. Following washing, the plates air dried at room temperature. The virus plaques formed in the MDBK cell cultures were quantified by manual counting. The inhibitory activities of test compounds were determined by calculating the percent (%) reduction in plaques as follows: % reduction in plaques=(number of plaques (drug-treated infected cells) divided by number of plaques (control (no compound) infected cells))×100. The data are presented in Table 3.

TABLE 3
Plaque Reduction Assay

| | EC₅₀ | | CC₅₀ | |
|---|---|---|---|---|
| Castanospermine | 110 ± 82 | μM | >1000 | μM |
| Interferon-α2b | 8.5 ± 4.4 | IU^{†} | >100 | IU |
| Ribavirin | 9.1 ± 6.3 | μM | 250 | μM |

^{†}IU = interferon resistance units

The EC₅₀ is the concentration of compound that inhibits 50% of viral plaque forming units as compared to an untreated control. These results further demonstrate, as did Example 2, that each of the tested compounds has a direct effect on viral proliferation.

### Example 4

### Synergism and Cytotoxicity of Castanospermine in Combination with INF-α2b or Ribavirin

A cytopathic assay was performed to determine the potential of castanospermine to act synergistically with interferon-α2b or ribavirin. A two-way combination assay was performed with average background- and color-corrected data in an inhibition-of-cytopathic effect (CPE) assay as described below. The two-way drug combinations were achieved by creating a “checkerboard,” with one drug being titrated horizontally and the other drug titrated vertically on MDBK cells infected with BVDV at an MOI of 0.01. The same approach was used on non-infected MDBK cells to look at the effect of the combination on the cytotoxicity of the drugs. Each two-way combination was performed twice. The EC₅₀ represents the concentration of compound that provides 50% protection of BVDV-induced cytotoxicity (CPE). The EC₅₀ data were analyzed using a MacSynergy® software program (gift from Dr. Mark Prichard, University of Alabama, Tuscaloosa, Ala.) to determine any synergistic effect (see, e.g., Ouzounov et al., supra; Buckwold et al., Antimicrob. Agents Chemother. 47:2293, 2003).

The EC₅₀ values of one antiviral derived from the addition of a second antiviral dose were plotted against the concentration of the second antiviral to create an isobole (dose pair), and all the isoboles were plotted in an isobologram to determine the presence of synergy, antagonism, or additivity. A line of additivity was plotted by connecting the monotherapy EC₅₀ values of each of the two test compounds (i.e., castanospermine and interferon or castanospermine and ribavirin). The line connecting the monotherapy EC₅₀ values represents the theoretical additivity effect values for two compounds. Isoboles of combination treatments below the additivity line indicate synergy, while isoboles above the additivity line indicate antagonism. The percent cytotoxicity (or % viability) of each compound alone and in combination on MDBK cells was also determined and used to calculate CC₅₀ values. The CC₅₀ of an antiviral is the dose that causes cytotoxicity in 50% of the cells when compared to untreated cells.

In combination with the isobolograms, MacSynergy™ II software was also used to obtain the percent synergy or percent of antagonism volumes for the double combination data. The calculated additive interactions were subtracted from the experimentally determined values to reveal the regions and corresponding drug concentrations at which a synergistic (indicated by positive % values) or antagonistic (indicated by negative % values) effect is observed. The horizontal plane at 0% inhibition represents the additivity (additive surface). The gradation in gray, a shift from light to dark, indicates the level of synergy. The 95% confidence intervals for the experimental dose-response surfaces were used to statistically evaluate the data.

Table 4 lists the concentration ranges for each compound that were used in each experiment.

TABLE 4
Concentration Range Tested

| Compound | Experiment 1 | Experiment 1 |
|---|---|---|
| Castanospermine (μM) | 3.7-300 | 6.25-100 |
| Ribavirin (μM) | 0.37-30 | 0.25-20 |
| Interferon α2b (IU/mL) | 0.40-250 | 0.08-50 |

Initially, as was done in Example 1, individual compound potency (EC₅₀) for inhibition of BVDV-induced cytopathic effects in MDBK cells was determined for each combination experiment (see Table 5). Minimal experiment-to-experiment variability in individual drug potency was observed.

TABLE 5
Protection of MDBK Cells from BVDV-Induced Cytotoxicity
(EC₅₀)

| Castanospermine | Interferon α2b | Ribavirin |
|---|---|---|
| 59 ± 16 μM | 19 ± 6 IU/mL | 4 ± 2 μM |

Dose Effect on EC₅₀

The EC₅₀ of castanospermine showed a dose-dependent decrease with increasing concentrations of interferon-α2b (Table 6). With an increasing concentration of interferon-α2b from 0 to 60 IU/mL, the EC₅₀ of castanospermine decreased from 52 μM to <1 μM with a about a 50-fold decrease at 20 IU/mL interferon-α2b (Table 6). Similarly, with an increasing concentration of castanospermine from 0 to 100 μM, the EC₅₀ of interferon-α2b decreased from 16 μM to <1 μM with about a 2-fold decrease at 11 μM of castanospermine (Table 6).

TABLE 6
Dose Effects on EC₅₀
Average Castanospermine EC₅₀ (μM)

| | | | | | | |
|---|---|---|---|---|---|---|
| Interferon-α2b added (IU/mL) | 0 | 0.7 | 2.2 | 6.7 | 20 | 60 |
| Castanospermine EC₅₀ (μM) | 52 | 39 | 19 | 12 | 1 | <1 |

Average Interferon-α2b EC₅₀ (IU/mL)

| | | | | | | |
|---|---|---|---|---|---|---|
| Castanospermine added (μM) | 0 | 1.2 | 3.7 | 11 | 33 | 100 |
| Interferon-α2b EC₅₀ (IU/mL) | 16 | 18 | 13 | 7 | 1 | <1 |

Double Combination Efficacy

A greater positive percent (synergy) volume is indicative of a synergistic effect. Values less than 25 μM/ml²% or 25 μM(IU)/ml(well)²% are insignificant; values between 25 and 50 μM/ml²% or 25 and 50 μM(IU)/ml(well)²% are considered minor but significant; values between 50 and 100 μM/ml²% or μM(IU)/ml(well)²% indicate moderate synergy that may be indicative of a significant synergistic effect in vivo; and values greater than 100 μM/ml²% or μM(IU)/ml(well)²% indicate strong synergy that is likely indicative of a significant synergistic effect in vivo.

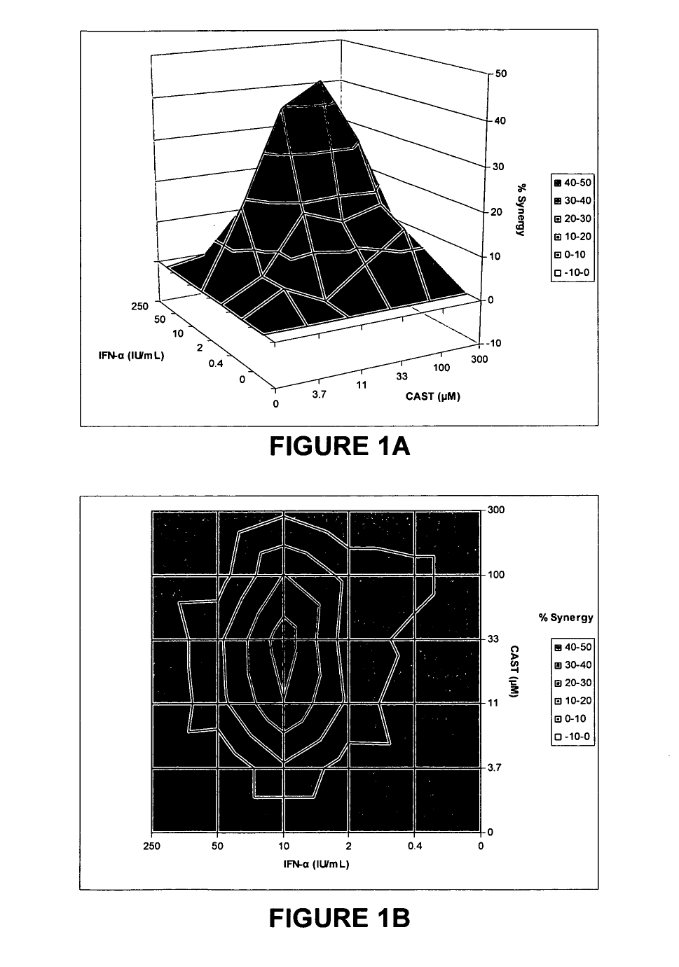

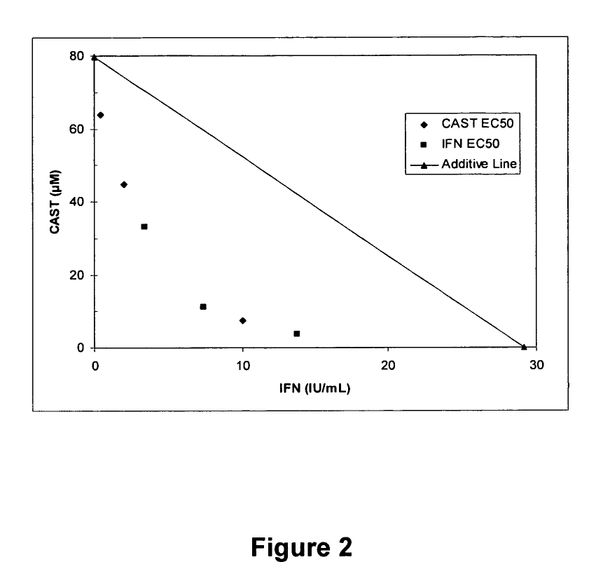

The castanospermine with interferon-α2b combination demonstrated strong synergy in efficacy against BVDV-infected MDBK cells (Table 7), and no significant antagonistic effects at any combination of concentrations tested (>−25 μM(IU/mL)%; any value less than −25 μM(IU/mL)% is a significant antagonistic effect). Synergistic peaks were located at castanospermine concentrations between 25 μM and 33 μM and an interferon-α2b concentration of 10 IU/mL (see FIG. 1). Analysis of the combination data using isobologram graphs confirms the strong synergy observed; for example, at 10 IU/mL of interferon-α2b, the EC₅₀ of castanospermine is reduced by >7-fold while a less than 2-fold reduction was expected (see FIG. 2).

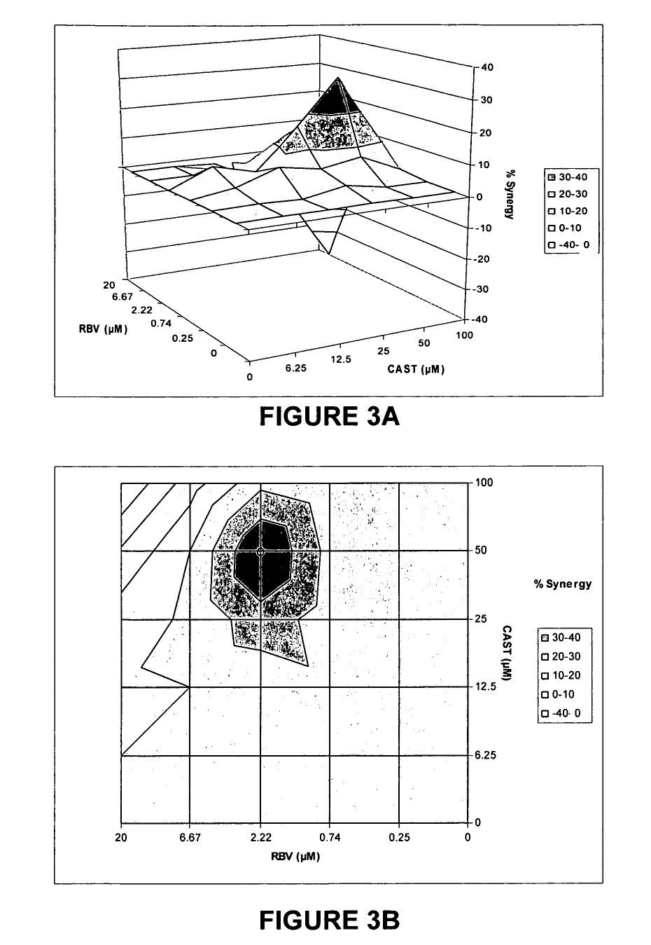

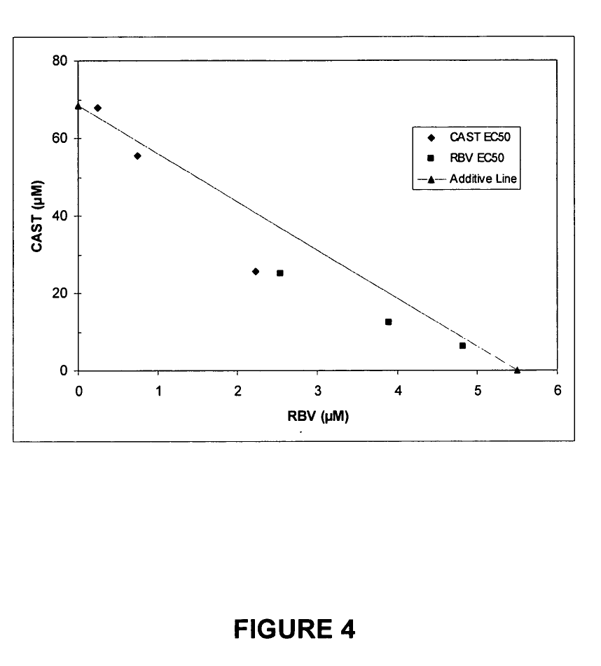

The castanospermine with ribavirin combination demonstrated moderate synergy in efficacy in BVDV-infected MDBK cells (Table 7). Synergistic peaks were located at castanospermine concentrations between 10 μM and 50 μM and ribavirin concentrations between 1 μM and 6 μM (see FIG. 3). The maximum percent synergy reached was between 22 μM²% and 31 μM²% (see FIG. 3). Antagonistic effects in efficacy (−145 μM²%) were observed at very high concentrations of the compounds—for example, antagonistic peaks occurred at a castanospermine concentration of 300 μM and a ribavirin concentration of 30 μM, which are unlikely to be relevant in vivo. The maximum percent antagonism reached was approximately −68 μM²%. Isobologram graphs derived from the combination of castanospermine with ribavirin also indicate synergy between castanospermine and ribavirin; for example, at about 2 μM ribavirin, the EC₅₀ of castanospermine is reduced by 2 to 3-fold while less than 2-fold reduction was expected (see FIG. 4).

The interferon α2b with ribavirin combination demonstrated moderate synergy in efficacy (68 μM(IU/mL)%) against BVDV-infected MDBK cells (Table 7). Similar volume of synergy has been reported in literature by Buckwold et al., 2003. Synergistic peaks were located at interferon-α2b concentrations between 2 IU/mL and 10 IU/mL and ribavirin concentrations between 0.7 μM and 3.3 μM (data not shown). The maximum percent synergy reached was between 20% and 30%. Antagonistic effects in efficacy (−102 μM(IU/mL)%) were also observed high concentrations of drugs, with antagonistic peaks occurring at interferon-α2b concentrations ≧50 IU/mL and ribavirin concentrations of ≧20 μM. The maximum percent antagonism reached was approximately −63 μM(IU/mL)%. Isobologram graphs derived from the combination of interferon α2b with ribavirin also indicate that there is synergy between interferon-α2b and ribavirin; for example at about 10 IU/mL interferon-α2b, the EC₅₀ of ribavirin is reduced by up to >6-fold, while about a 2-fold reduction was expected (data not shown).

TABLE 7
Efficacy Synergy Volume of Double Combination Treatment

| Combination | Efficacy Synergy(95% CI) |
|---|---|
| Castanospermine/IFN-α2b (μM(IU/mL) %) | 231 ± 52 |
| Castanospermine/Ribavirin (μM² %) | 97 ± 11 |
| Ribavirin/IFN-α2b (μM(IU/mL) %) | 68 ± 1 |

CI = Confidence Interval;
IFN-α2b = Interferon α2b

Double Combination Cytotoxicity

A greater negative percent (antagonist) volume is indicative of the combination having lesser impact on cytotoxicity. A value of less than −25 μM(IU/mL)% is considered a significant antagonistic effect (i.e., no significant cytotoxicity).

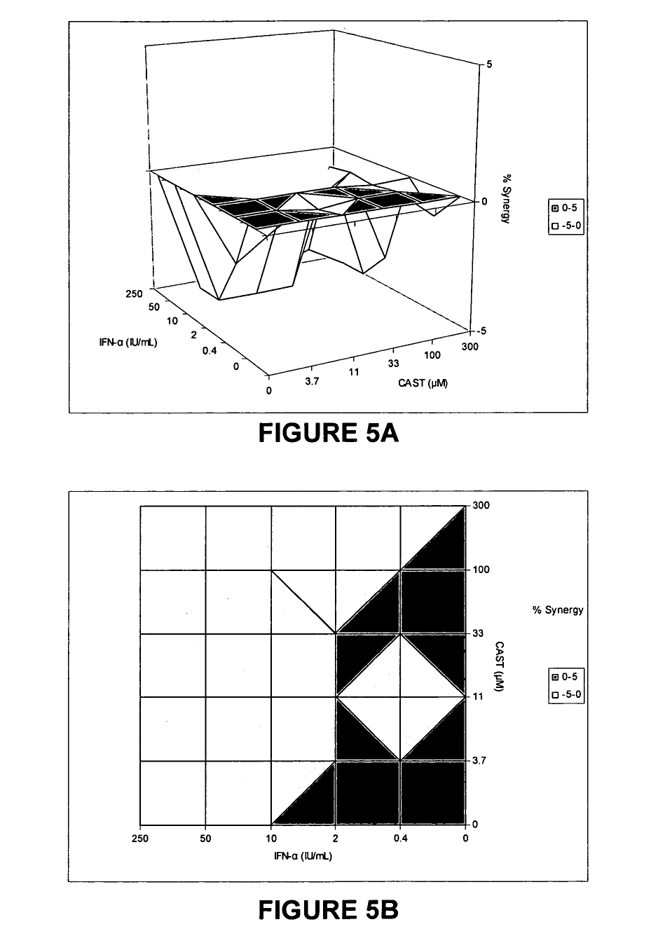

The castanospermine with interferon-α2b combination demonstrated moderate antagonistic effects in cytotoxicity (−63 μM(IU/mL)%) in uninfected MDBK cells, while no synergistic effects (>25 μM(IU/mL)%) were observed (Table 8). Antagonistic troughs were located at castanospermine concentrations between 50 μM and 100 μM and interferon-α2b concentrations greater than 0.4 IU/mL (see FIG. 5). The maximum percent antagonism reached was between −9% and −17%.

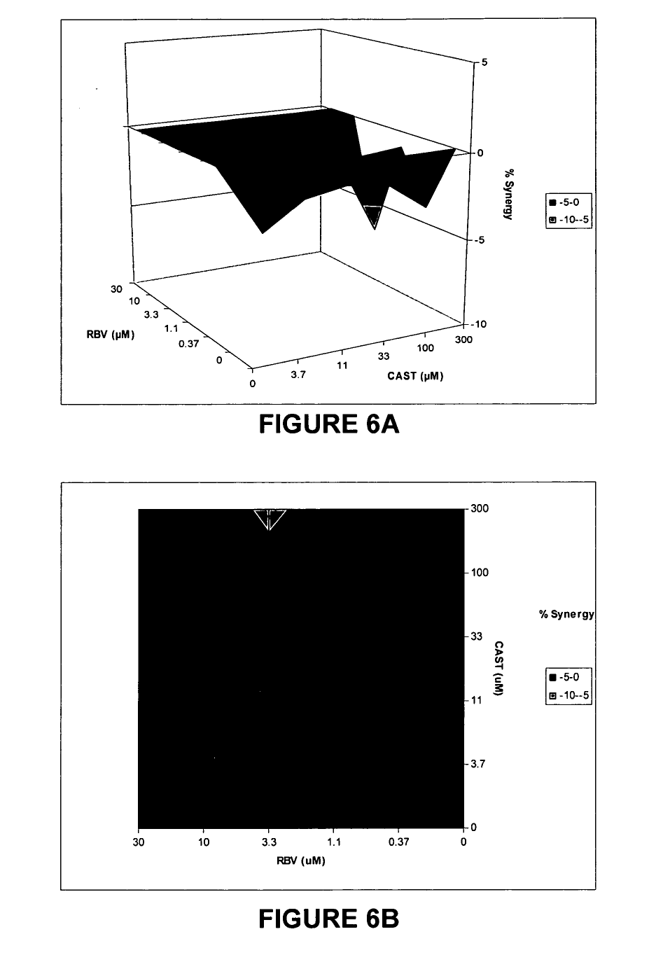

The castanospermine with ribavirin combination also demonstrated moderate antagonistic effects in cytotoxicity (−46 μM²%) in uninfected MDBK cells, while no synergistic cytotoxic effects (>25 μM²%) were observed (Table 8). Antagonistic troughs were located at castanospermine concentrations of greater than 20 μM and ribavirin concentrations of approximately 3 μM. The maximum percent antagonism reached was between −6% and −7% (see FIG. 6).

The interferon α2b with ribavirin combination demonstrated moderate antagonistic effects in cytotoxicity with an average of −83 μM (IU/mL)% in uninfected MDBK cells, while no significant synergistic effects (>25 μM²%) were observed (Table 8). Antagonism was quite uniform throughout the concentration ranges of the two antivirals with no region experiencing significantly higher antagonism than other areas (data not shown). The maximum percent antagonism reached was between −8% and −18%.

TABLE 8
Cytotoxicity Antagonism Volumes of Double Combination Treatment

| | Cytotoxicity (95% CI) | |
|---|---|---|
| Combination | Synergy | Antagonism |
| Castanospermine/IFN-α2b (μM(IU/mL)%) | 0 | −63 ± 10 |
| Castanospermine/Ribavirin (μM² %) | 0 | −46 ± 13 |
| Ribavirin/IFN-α2b (μM(IU/mL)%) | 6 ± 1 | −83 ± 18 |

CI = Confidence Interval;
IFN-α2b = Interferon α2b

Overall, combinations of castanospermine with interferon-α2b were strongly synergistic (volumes of synergy >100 (IU/mL)μM %). Combinations of castanospermine with ribavirin showed a more moderate synergy (between 25 μM²% and 100 μM²%). Strong antagonistic volumes observed in the combinations occurred at high drug concentrations and are unlikely to be relevant in vivo. The cytotoxic volumes for the combinations were either antagonistic, indicating that the combinations had no significant impact on their cytotoxicity. This antagonism in cytotoxicity may indicate that the combination can reduce the individual cytotoxicities of each compound. A dose-dependent reduction in EC₅₀ of castanospermine (up to >52-fold) was observed upon the addition of increasing concentrations of interferon-α2b. This decrease in EC₅₀ was more pronounced with the addition of increasing concentrations of ribavirin. The combinations did not increase the cytotoxicity of interferon-α2b or ribavirin. These data indicate that the combination of castanospermine with interferon α2b and the combination of castanospermine with ribavirin could be beneficial for the treatment of HCV-infected patients.

### Example 5

### Synergism and Cytotoxicity of Castanospermine in Combination with Amantadine or NB-DNJ

A cytopathic assay was performed to determine the potential of castanospermine to act synergistically with amantadine or NB-DNJ. A two-way combination assay was performed with average background- and color-corrected data in an inhibition-of-cytopathic effect (CPE) assay as described below. The two-way drug combinations were achieved by creating a “checkerboard,” with one drug being titrated horizontally and the other drug titrated vertically on MDBK cells infected with BVDV at an MOI of 0.05. The same approach was used on non-infected MDBK cells to look at the effect of the combination on the cytotoxicity of the drugs. Each two-way combination was performed twice. The EC₅₀ represents the concentration of compound that provides 50% protection of BVDV-induced cytotoxicity (CPE). The EC₅₀ data were analyzed using a MacSynergy® software program (gift from Dr. Mark Prichard, University of Alabama, Tuscaloosa, Ala.) to determine any synergistic effect (see, e.g., Ouzounov et al., supra; Buckwold et al., Antimicrob. Agents Chemother. 47:2293, 2003). Also tested was M-1914 (a non nucleoside HCV inhibitor of HCV polymerase that does not inhibit BVDV) as a negative control.

Individual potencies (EC₅₀) of each of the drugs for inhibition of BVDV-induced cytopathic effects in MDBK cells were determined for each combination experiment (see Table 9).

TABLE 9
Protection of MDBK Cells from BVDV-Induced Cytotoxicity (EC₅₀)

| Castanospermine | Amantadine | M-1914 | NB-DNJ |
|---|---|---|---|
| >100 | 400.4 | >50 | >500 |

Double Combination Efficacy Studies

Combination of castanospermine with amantadine showed moderate synergy (volumes of synergy 60.7 μM²%) in inhibiting the cytopathic effect of BVDV in MDBK cells. Combinations of castanospermine with NB-DNJ or M-1914 were not significantly synergistic (volumes of synergy <25 μM²%) in inhibiting the cytopathic effect of BVDV in MDBK cells. No significant antagonism was observed between castanospermine and amantadine, castanospermine and NB-DNJ, or castanospermine and M-1914 combinations (volumes of synergy >−25 μM²%) (Table 10).

TABLE 10
Efficacy Synergy Volumes of Combination Treatment

| Combination | Efficacy Synergy (μM² %) (95% CI) |
|---|---|
| Castanospermine-Amantadine | 60.7 |
| Castanospermine-M-1914 | 6 |
| Castanospermine-NB-DNJ | 16.3 |

CI = Confidence Interval;
range tested: Castanospermine = 33.3-0.4 μM;
M-1914 = 50-3.12 μM;
Amantadine = 250-31.3;
NB-DNJ = 250-31.3

Double Combination Cytotoxicity Studies

The cytotoxicity of castanospermine in combination with amantadine, M-1914, or NB-DNJ was determined in uninfected MDBK cells. The cytotoxic volumes for the double combinations were additive for the double combinations of castanospermine with amantadine, M-1914, or NB-DNJ (volumes of synergy <25 μM²%). No significant antagonism was observed for the combination castanospermine with NB-DNJ (volume of synergy >−25 μM²%), while moderate antagonism was observed with the combinations castanospermine with amantadine (volume of synergy −40.1 μM²%) or castanospermine-M-1914 (volume of synergy −26.3 μM²%), indicating that addition of castanospermine to amantadine or M-1914 can reduce their expected toxicities.

TABLE 11
Cytotoxicity Antagonism Volumes of Combination Treatment

Cytotoxicity Volumes
(μM² %) (95% CI)

| Combination | Synergy | Antagonism |
|---|---|---|
| Castanospermine-Amantadine | 1.3 | −40.1 |
| Castanospermine-M-1914 | 0 | −26.3 |
| Castanospermine-NB-DNJ | 8.1 | −1.7 |

Those skilled in the art will recognize, or be able to ascertain, using no more than routine experimentation, many equivalents to the specific embodiments of the invention described herein. Such equivalents are intended to be encompassed by the following claims.

We claim the following:

1. A method for treating a Flaviviridae infection comprising administering to a subject castanospermine, or a pharmaceutically acceptable salt thereof, and an agent that alters immune function.

2. A method for treating a Flaviviridae infection comprising administering to a subject castanospermine, or a pharmaceutically acceptable salt thereof, and an agent that alters replication of a virus of the Flaviviridae family.

3. The method according to either claim 1 or claim 2 wherein the subject is a human.

4. The method according to either claim 1 or claim 2 wherein the virus of the Flaviviridae family is a member of the genus Flavivirus.

5. The method according to either claim 1 or claim 2 wherein the virus of the Flaviviridae family is a member of the genus Pestivirus.

6. The method of claim 4 wherein the Flavivirus is a Hepacivirus, wherein the Hepacivirus is Hepatitis C virus (HCV).

7. The method according to claim 1 wherein the agent that alters immune function is an interferon.

8. The method according to claim 7 wherein the interferon is an interferon-α.

9. The method according to claim 8 wherein the interferon-α is pegylated.

10. The method according to claim 1 wherein castanospermine and the agent that alters immune function are administered sequentially.

11. The method according to claim 10 wherein the agent that alters immune function is administered before castanospermine.

12. The method according to claim 10 wherein castanospermine is administered before the agent that alters immune function.

13. The method according to claim 1 wherein castanospermine and the agent that alters immune function are administered concurrently.

14. The method according to claim 1 wherein castanospermine and the agent that alters immune function are admixed as a single composition and administered concurrently.

15. The method according to claim 1 comprising administering to a subject (a) a composition comprising castanospermine and a pharmaceutically acceptable carrier and (b) a composition comprising the agent that alters immune function and a pharmaceutically acceptable carrier.

16. The method according to claim 2 wherein the agent that alters viral replication is ribavirin.

17. The method according to claim 2 wherein the agent that alters viral replication is interferon-α.

18. The method according to claim 2 wherein castanospermine and the agent that alters viral replication are administered sequentially.

19. The method according to claim 18 wherein the agent that alters viral replication is administered before castanospermine.

20. The method according to claim 18 wherein castanospermine is administered before the agent that alters viral replication.

21. The method according to claim 2 wherein castanospermine and the agent that alters viral replication are administered concurrently.

22. The method according to claim 2 wherein castanospermine and the agent that alters viral replication are admixed as a single composition and administered concurrently.

23. The method according to claim 1 wherein castanospermine and the agent that alters immune function interact synergistically.

24. The method according to claim 2 wherein the castanospermine and the agent that alters viral replication interact synergistically.

25. A method for treating Flaviviridae infection comprising castanospermine in combination with an agent selected from:
(a) a compound that inhibits infection of cells by Flaviviridae;
(b) a compound that inhibits the release of viral RNA from the viral capsid or inhibits the function of Flaviviridae gene products;
(c) a compound that alters Flaviviridae replication;
(d) a compound that alters immune function;
(e) a compound that alters symptoms of a Flaviviridae infection; and
(f) a compound for treating Flaviviridae-associated infections.

26. The method according to claim 25 wherein the compound that alters immune function is an interferon.

27. The method according to claim 25 wherein the interferon is interferon-α.

28. The method according to claim 27 wherein the interferon is pegylated interferon-α.

29. The method according to claim 25 wherein the compound that alters Flaviviridae viral replication is ribavirin or interferon-α.

30. The method according to claim 25 wherein the Flaviviridae-associated infection is a hepatitis B viral (HBV) infection or a retroviral infection.

31. The method according to claim 30 wherein the retroviral infection is a human immunodeficiency virus infection (HIV).

32. A method for treating a Flaviviridae infection comprising administering to a subject castanospermine, or a pharmaceutically acceptable salt thereof, and an interferon.

33. The method according to claim 32 wherein the interferon is interferon-α.

34. The method according to claim 32 wherein the interferon is pegylated interferon-α.

35. The method according to claim 32 wherein castanospermine and the interferon interact synergistically.
